(19) United States
(12) Patent Application Publication
(10) Pub. No.: US 2019/0218575 A1
(43) Pub. Date: Jul. 18, 2019

(54) SELF-ITERATING EXOSOMAL VECTOR FOR EFFECTIVE BIOMOLECULAR TRANSFERS

(71) Applicant: William Douglas Meadow, Jr., Jacksonville, FL (US)

(72) Inventor: William Douglas Meadow, Jr., Jacksonville, FL (US)

(21) Appl. No.: 16/366,806

(22) Filed: Mar. 27, 2019

Related U.S. Application Data
(63) Continuation of application No. 15/351,171, filed on Nov. 14, 2016, now abandoned.
Application No. 15/351,171 is a continuation of application No. 14/225,070, filed on Mar. 25, 2014, now abandoned.

(51) Int. Cl. C12N 15/88; C12N 7/00; C12N 15/85; C12N 15/86

(52) U.S. Cl. CPC C12N 15/88; C12N 7/00; C12N 2710/24143; C12N 15/86; C12N 2810/40; C12N 15/85

(57) ABSTRACT

The present invention provides for a process for transferring biomolecules such as polynucleotides and protein from cell to cell, eventually resulting in the transport of a biomolecular cargo throughout the entirety of one or more of a cell culture, tissue, organ, organ system, or organism. The method also includes translocation of an SIF construct complex into a cell.

### BACKGROUND OF THE INVENTION

In the subject area of transfection for research, experimental, medical, and diagnostic purposes, viral vectors have traditionally been limited by immune reactions to the mechanism of DNA transfer, while non-viral vectors have traditionally been limited in their effectiveness and proliferation.

Exosomes are intercellular vesicles created by the fusion of a multi-vesicular body, a vesicle containing one or more intra-lumenal vesicles (ILVs), with the plasma membrane of a cell. Their lipid, protein, and RNA compositions differ substantially from those of their donor cells, indicating that there exist selective uptake mechanisms for transport into the ILVs that later become exosomes. Candidate factors for ILV translocation include lipids such as ceramide and proteins such as the ESCRT complexes, among other biomolecules.

### SUMMARY OF THE INVENTION

The present invention describes a novel process that results in the transfer of biomolecules such as polynucleotides and protein from cell to cell, eventually resulting in the transport of a biomolecular cargo throughout the entirety of one or more of a cell culture, tissue, organ, organ system, or organism.

A Self-Iterating Factor (SIF) is a DNA-binding fusion protein that has an affinity for factors involved in ILV translocation such that the DNA attached to the SIF is incorporated into an ILV, which later becomes an exosome. According to the present invention, an exosome then fuses with a target cell, releasing the genetic construct that can be defined by one of many natural bioprocesses and then placed in the SIF, which serves as a robust transport method for biomolecular transfer such that desired bioprocesses can occur in the target biological system. These processes involve the alteration of one or more of: the genome, the transcriptome, and the proteome.

Using its species-matched origin of replication, the genetic construct replicates before the SIF-bound copy of the construct is incorporated into yet another ILV, restarting the self-iterating cycle of transfection with the genetic construct. Specifically, the present invention teaches that the genetic construct codes for the SIF (among other gene products), such that new copies of the SIF-construct complex can be regenerated in the cytoplasm as time passes. Because the genetic construct is DNA shuttled between cells via an exosome, this DNA is known as exosomal shuttle DNA (esDNA). Unlike previously known processes, the process taught by the present invention does not repeat indefinitely, as the gene encoding the SIF contains signals for methylation or other nucleotide alterations that will decrease gene expression over time.

A self-iterating exosomal vector therefore combines the proliferation of a viral vector with the immune non-recognition found in non-viral vectors due to the fact that the exosomes are derived from the target organism.

The essence of the invention is that exosomes, intercellular transport vesicles, can provide a self-iterating transfer method for sending biomolecules such as nucleic acids and protein from cell to cell, particularly DNA. This culminates in the transfer of the biomolecular cargo throughout the entirety of a cell culture, tissue, organ, organ system, or organism.

Exosomes arise from endosomal patches of the plasma membrane budding outward as well as from intra-lumenal vesicles of multi-vesicular bodies being released when the outer membranes of multi-vesicular bodies fuse with the plasma membrane. A genetic construct encoding a DNA-binding fusion protein (self-iterating factor, or SIF) with an affinity for factors involved in intra-lumenal vesicle translocation is thus able to facilitate its own sorting into an exosome for intercellular movement when the SIF is bound to the construct. As the construct codes for more copies of the SIF and can replicate by borrowing DNA replication enzymes from its host cell, this process of exosomal movement of the genetic construct is self-perpetuating. Likewise, with the use of other protein-encoding genes on the genetic construct, the self-iterating spread of a protein by means of exosomes can also be facilitated.

### DESCRIPTION OF FIGURES

FIG. 01 demonstrates both phases of process 2, in which Calcium lonophore (A23187); hereafter referred to as A23187 in the description and figures, is added to a cell in order to induce exosome release. In the second phase, an exosome containing RNA and protein but not DNA is shown. Process 1, not shown, may refer to a standard method of preparing an untransfected cell culture to begin an experiment with.

FIG. 02 illustrates both phases of process 10, in which A23187 causes a recently released endosome to fold inward, creating an ILV. Process 11 also takes place at the site of ILV formation.

FIG. 03 also shows process 11, in which A23187 works with a SIF to cause a genetic construct to enter an ILV. It also shows processes 15 and 17, where type 1 and type 2 exosomes are released at the plasma membrane, respectively.

FIG. 04 depicts the three phases of process 4, in which a liposome containing a SIF-construct complex fuses with an exosome to create an exosome loaded with a SIF-construct complex.

FIG. 05 demonstrates processes 6-9, in which exosomes loaded with SIF-construct complexes reach target cells and deliver their SIF-construct complexes. They may reach their target cells by being introduced to a cell culture (process 6) if the self-iterating cycle is being started. Alternatively, the exosomes may reach their target cells by movement through the extracellular space (process 7) if the self-iterating cycle is taking place. In processes 8 and 9, cells which have received SIF-construct complexes experience replication of the SIFs and constructs.

FIG. 06 illustrates processes 19-22, in which exosomes are brought into contact with liposomes containing the genetic construct. After fusion of the membranes, the result is exosomes containing the genetic construct. These exosomes can then be added to the target cells such that fusion of the membranes results in cells containing the genetic construct.

FIG. 07 shows the 22 processes involved with the patent and depicts their relation to the previously mentioned FIGS. 01-06. Each process relates to a specific figure, with each figure showing multiple processes. Specifically, the self-iterating cycle is FIGS. 02-05 repeating themselves. FIG. 07 references FIGS. 01-06, as it places all of them in a single context.

### DETAILED DESCRIPTION

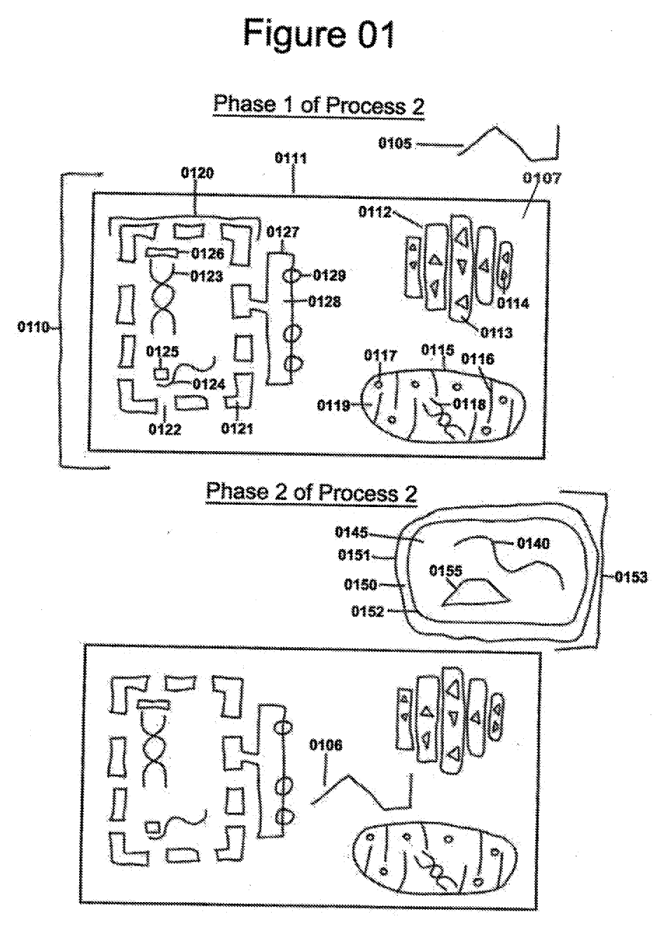

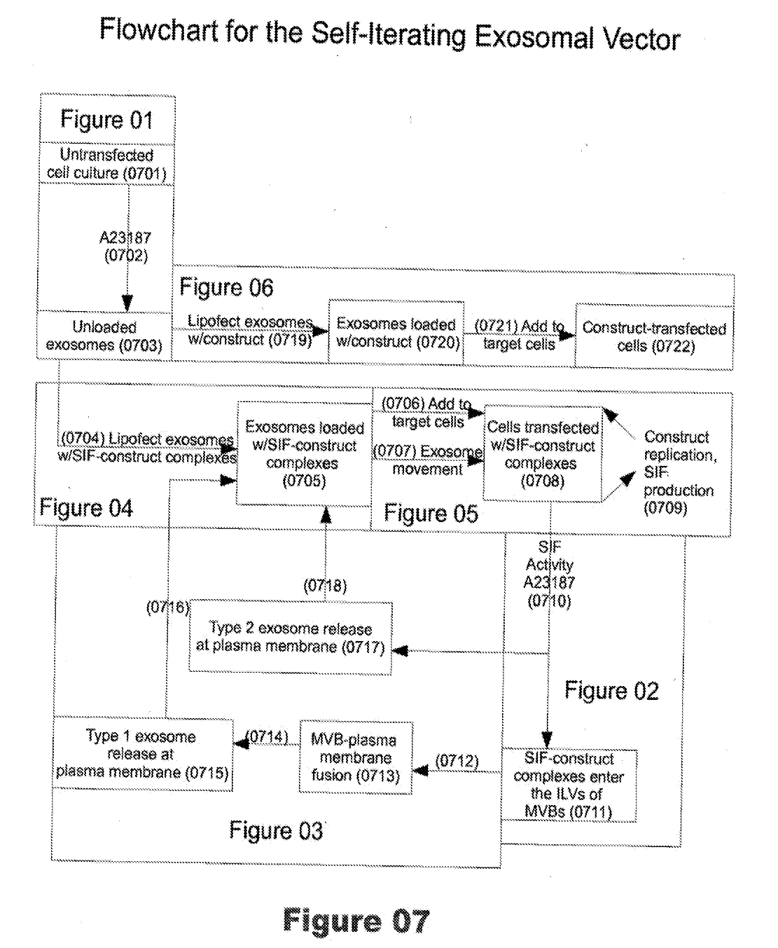

FIG. 01 represents processes 0701 through 0703, as shown in FIG. 07. In the following description, process numbers in the individual figures may appear as numbers from 01-22. These numbers may also be referred to with a prefix of “07” preceding the number of the process since in FIG. 7, the reference numbers are presented in this manner. Process 0701 is to prepare an untransfected cell culture. A single cell [0110] is shown in both phase 1 and 2 of process 0702. In phase 1, the various components of this typical eukaryotic cell [0110] are labeled. The plasma membrane [0111] surrounds the cytosol [0107], which contains the golgi apparatus [0112], a mitochondrion [0115], and the nucleus [0120]. Outside the cell is a sample molecule of calcium ionophore (A23187) [0105].

The golgi apparatus [0112] consists of phospholipid bilayer membranes [0112] enclosing several membrane-bound vesicles [0113]. Within the internal spaces of the golgi apparatus, a variety of proteins exist [0114].

The mitochondrion consists of two closely packed phospholipid bilayer membranes [0115] enclosing a small intermembrane space and the interior mitochondrial matrix [0119]. The interior membrane is shaped into folds called cristae [0116] that point inward within the mitochondrial matrix [0119]. The mitochondrial matrix contains a mtDNA (mitochondrial DNA) plasmid [0118], ribosomes [0117], and proteins (not shown).

The nucleus [0120] consists of the nuclear envelope [0121], the nuclear pores [0122], DNA molecules [0123], among other entities. Also included within the nucleus are the RNA molecules [0124] and protein components [0125] of the nucleolus, as well as enzymes such as RNA polymerase [0126]. Outside of the nucleus, the rough endoplasmic reticulum [0127] has an internal lumen [0128] and ribosomes on its exterior [0129].

Molecules of A23187, such as the one shown [0105], enter the cell during process 0702 [0106]. According to research by Valadi et al., placing HMC-1 cells in a 2 uM solution of A23187 for 30 minutes stimulates exosome release. Thus, A23187 is used as the sample means by which exosome release takes place.

A sample exosome [0153] is shown in phase 2 of process 0702, although this also represents the beginning of process 0703, in which exosomes containing no DNA (unloaded exosomes) are generated. The interior of the exosome [0145] is surrounded by a phospholipid bilayer [0150] with an inner [0152] and outer [0151] component. Within the exosome exist molecules of RNA [0140] and protein [0155].

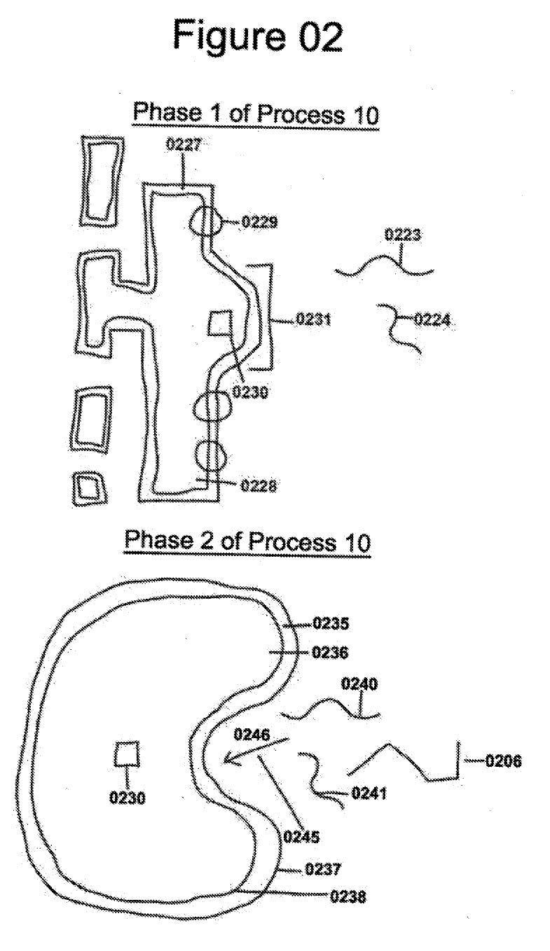

FIG. 02 shows how intra-lumenal vesicles (ILVs), the predecessors of type 1 exosomes, begin to form. Type 1 exosomes correspond to the ILVs of multi-vesicular bodies (MVBs) upon fusion of the outer membrane of a MVB with the plasma membrane of a cell, whereas type 2 exosomes form from endosomal patches of the plasma membrane budding outwards. As explained in FIG. 07, FIG. 02 corresponds to processes 0710 and 0711. In phase 1 of process 0710, the rough endoplasmic reticulum [0227] begins to bud off an endosome [0231] containing protein [0230]. As previously shown, the rough endoplasmic reticulum has an internal membrane-bound lumen [0228] and has ribosomes on its exterior [0229]. In the nearby cytosol, RNA molecules are present [0223, 0224].

In phase 2 of process 0710, A23187 [0206] facilitates the infolding [0245] of the endosomal membrane [0235] in order to initiate the creation of an ILV. The example protein [0235] within the endosomallumen [0236] represents the many proteins that may exist within the endosomallumen [0236]. The endosomal membrane [0235] is a phospholipid bilayer with an inner [0238] and outer [0237] component. RNA molecules [0240, 0241] from the cytosol enter the ILV [0246], as shown. When the ILV becomes an exosome, these RNA molecules [0240,0241] are referred to as exosomal shuttle RNA (esRNA).

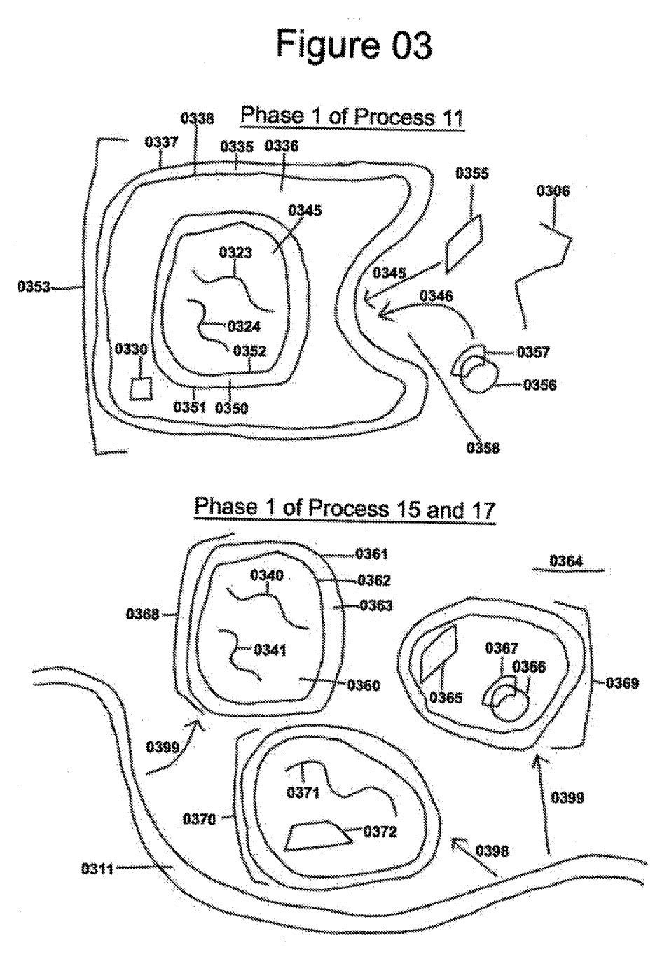

FIG. 03 corresponds to processes 11-18. In particular, process 0711 is related to both FIG. 02 and FIG. 03, as both illustrate cellular cargo [0240/0241, 0355/0356/0357] entering an ILV [0246,0345, 0346]. In FIG. 03, A23187 [0306] facilitates the infolding [0358] of the external membrane of the MVB [0335], which is a phosopholipid bilayer [0337, 0338]. Protein from the cytosol [0355] may then enter [0345] the newly forming ILV [0358]. In the case of the genetic construct—pGFP in this example—[0356], the SIF [0357] facilitates the entry [0346] of the SIF-construct complex [0356, 0357] into the newly forming ILV

The MVB may already contain protein [0330] inside of its intermembrane space [0336], as shown. The phospholipid bilayer [0351, 0352] membrane [0350] of the already-formed ILV [0345] surrounds the RNA molecules [0323, 0324] that will eventually become esRNAs [0340, 0341]. The above information relates to phase 1 of process 0711.

In process 12, the MVB [0335] moves through the cytosol to reach the plasma membrane [0311], where it fuses (process 0713) and releases its contents [0368, 0369]—two ILVs that become exosomes in processes 14 and 15. Alternatively, endosomal patches of the cellular membrane [0311] may spontaneously release type 2 exosomes [0370] in process 0717. In this way, the second diagram of FIG. 03 represents processes 15 and 17. In processes 16 and 18, the two types of exosomes [0368, 0369, 0370] move outward into the extracellular space [0364]. Both types of exosomes [0368, 0369, 0370] have a phospholipid bilayer [0361, 0362] membrane [0363] enclosing an exosomallumen [0360]. Exosomes [0368, 0369, 0370] may contain both esRNA [0340, 0341, 0371] and protein [0365, 0372]. As shown, the content of any single exosome [0368, 0369, 0370] may vary significantly.

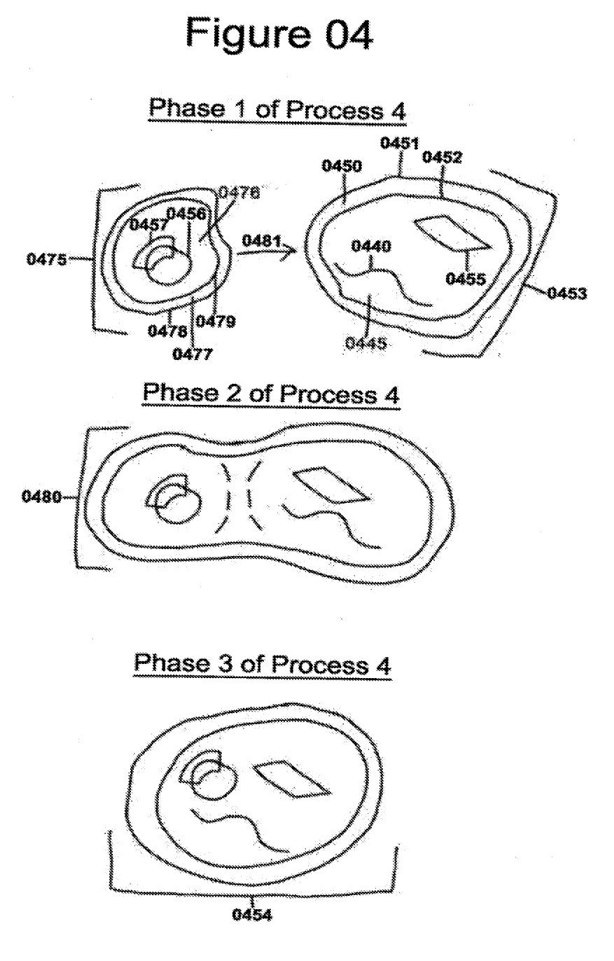

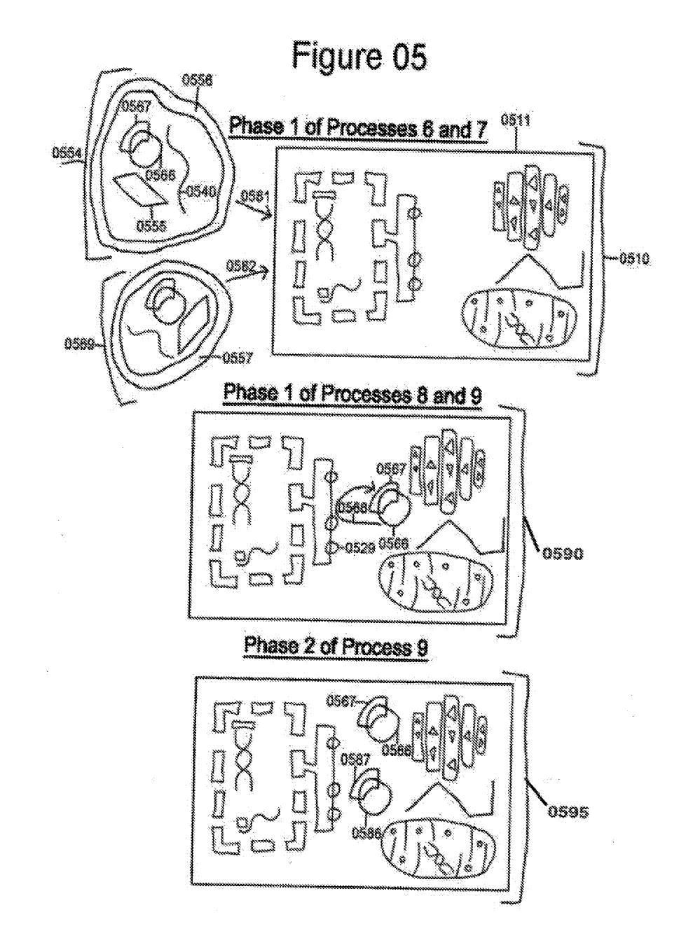

An important feature of FIG. 03 is that in process 0711, the SIF [0357, 0367] facilitates the entry of a cytosolic genetic construct [0356] into a newly forming ILV [0358]. Through processes 12-16, the ILV containing the SIF-construct complex [0356/0357, 0366/0367] eventually becomes an exosome [0368] loaded with the SIF-construct complex [0366, 0367]. FIGS. 04 and 05 depict how an exosome may eventually deliver its SIF-construct complex [0366, 0367] to another cell, where it may replicate in process 0709, thus creating a self-iterating cycle (FIGS. 02-05) of DNA transfer. This ultimately has the potential to become a vector which will promote the spread of a genetic construct throughout the entirety of a multi-cellular organism in a non-viral manner.

FIG. 04 illustrates the three phases of process 0704, which result in process 0705, the generation of exosomes [0454] loaded with SIF-construct complexes [0456, 0457]. Using a lipofection reagent, liposomes [0475] which contain SIF-construct complexes [0456, 0457] in their lumen [0476] are generated. These liposomes [0475] have membranes [0477] which are phospholipid bilayers with inner [0479] and outer [0478] components. As shown in FIG. 01, the addition of A23187 to cells can result in the creation of exosomes [0153, 0453] which are not loaded with DNA. The addition of these liposomes constitutes phase 1 of process 0704. However, lipofection of exosomes [0480] in phase 2 of process 0704 can remedy this situation. As this occurs [0480], the SIF-construct complexes [0456, 0457] enter the exosomes During phase 2, the outer layers [0478, 0451] and the inner layers [0479, 0452] of the liposome and exosome fuse such that the two phospholipid bilayers [0477, 0450] become one. In this way, the cargo of the liposome [0456, 0457] and exosome [0440, 0445] become united within one biological vesicle [0480] during phase 2. In phase 3 of process 0704, the fusion is complete and the result [0454] culminates in process 0705, which is the generation of composite exosomes [0454] containing SIF-construct complexes [0456, 0457].

FIG. 05 corresponds to processes 6-9. Exosomes [0554, 0569] generated in process 0705 are shown to the left of a cell [0510]. In order to start the self-iterating cycle (FIGS. 02-05), exosomes [0454] containing SIF-construct complexes [0554] may be added to cells [0581] in process 0706. If the cycle (FIGS. 02-05) is already in progress, then exosomes [0569] from other cells [0311] may reach [0582] target cells [0510] in process 0707 by means of exosome movement. As previously shown, exosomes can contain both RNA [0540] and protein [0555] content. Processes 6 and 7 culminate in the fusion [0581, 0582] of exosomal membranes [0556, 0557] with the plasma membranes [0511] of target cells [0510].

Process 0708 in FIG. 05 refers to the generation of cells transfected [0590] with SIF-construct complexes [0566, 0567], the restart step in the self-iterating cycle (FIGS. 02-05). This, as previously stated, occurs as exosomes [0554, 0569] deliver their SIF-construct complexes [0566, 0567] to the target cells [0510, 0590] through exosome movement [0581, 0582] and membrane fusion [0556, 0557, 0511]. This also represents the first phase of process 0709, in which SIF-construct complexes in cells replicate. Alternatively, process 0708 may be reached from process 0701 if the cells are lipofected with SIF-construct complexes.

Phase 2 of process 0709 in FIG. 05 refers to the replication of the SIF-construct complexes [0566, 0567]. As previously mentioned, the genetic construct [0566] codes for the SIF [0567], among other gene products. Over time, the presence of the genetic construct [0566] in the cytosol will lead to the transcription of the SIF mRNA and the translation [0568] of this mRNA into the SIF protein [0567] at a ribosome [0529]. Furthermore, the SIF [0567], like most DNA-binding proteins, has a variable affinity for its binding site and can dissociate at times. When the genetic construct [0566] is not bound to its SIF [0567], it is unlikely to be taken into an ILV and transported to another cell via exosomal shuttling. This allows for the DNA replication enzymes of the host cell [0510, 0590, 0595] to produce a new copy of the genetic construct [0586]. In this way, new copies of the SIF [0587] and genetic construct [0586] can be produced. In order to prevent this process from continuing indefinitely, the SIF-encoding gene on the genetic construct [0566, 0586] contains signals for methylation that will reduce the expression [0568] of that particular gene over time. Ideally, SIF production [0568] will halt when nearly every cell [0590, 0595] in the culture or multi-cellular organism has at least one copy of the genetic construct [0566, 0586] such that expression of the genetic construct [0566, 0586, 0529] will be nearly universal. This part of FIG. 05, process 0709, completes the self-iterating cycle, which is defined as FIGS. 02-05 repeating until expression of the genetic construct's SIF-encoding gene [0566, 0568, 0567] declines significantly.

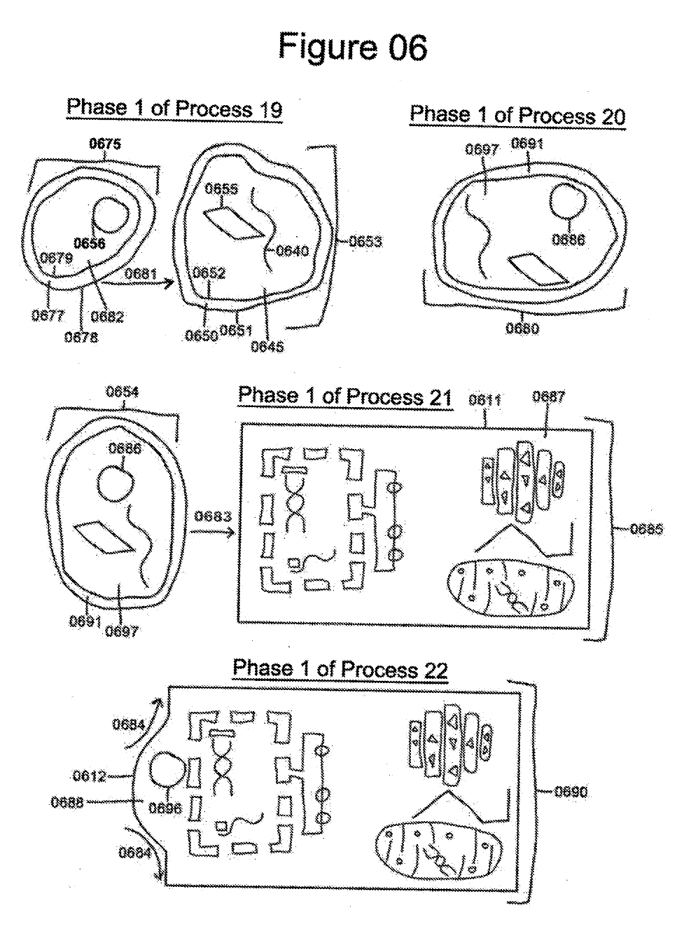

FIG. 06 represents the one-time variant of the exosomal vector [0654]. Specifically, the sequence of processes 19 through 22 results in the transfecting [0683] of target cells [0685] with copies of the genetic construct [0656]. In phase 1 of process 0719, the lipofection of exosomes [0653] by means of liposomes [0675] carrying the genetic construct [0656] is shown. As previously explained, both liposomes [0675] and exosomes [0653] have membranes [0677, 0650] which are phospholipid bilayers with inner [0679, 0652] and outer [0678, 0651] components. Also, exosomes typically contain both RNA [0640] and protein [0655] content. In process 0719, the fusion [0681, 0691] of the membranes [0677, 0650] of the liposomes [0675] and exosomes [0653] combines the two interiors [0682, 0645] into one new lumen [0686]. This brings the overall procedure to phase 1 of process 0720, the generation of newly created exosomes [0680] loaded with the genetic construct [0686] in their interior [0697]. In processes 21 and 22, these exosomes [0654] loaded with the genetic construct [0686] are added [0683] to the target cells [0685] such that the two plasma membranes [0691, 0611] rearrange themselves [0684] and fuse together into a single membrane [0612]. As this occurs, the exosomallumen [0697] becomes one with the cytosol [0687, 0688]. Furthermore, this action delivers the genetic construct [0686] in the lumen [0697] of the exosome [0654] into the cytosol [0687, 0688] such that it [0686, 0696] now resides in the cytosol [0688] of the composite cell [0690].

FIG. 07 is the flowchart that describes all 22 of the above processes in a single page. For a one-time transfection, one can simply follow FIGS. 01 and 06 [0153; 0653;0680/0654, 0681, 0683,0696]. The sample chemical used for exosome release, A23187 [0105/0106, 0206], is used in both FIG. 01 and FIG. 02. In FIGS. 02 and 03, the first steps [0231, 0206, 0246] leading to exosome release [0368, 0369, 0370] occur as part of a self-iterating cycle (FIGS. 02-05). process 0711 [0346], in which SIF-construct complexes [0356, 0357] enter the ILVs [0358] of MVBs [0353], is related to both FIGS. 02 and 03, as both feature the activity [0245, 0358] of A23187 [0206, 0306]. In FIG. 03, both types of exosomes [0368/0369, 0370] are shown outside [0364] of a cell [0311] just as process 0705 [0368, 0369, 0370] is reached from processes 15/16 [0399] and 17/18 [0398].

In FIG. 04, process 0704 [0481], the alternative method of reaching process 0705 [0480, 0454], is shown. This serves as the connection between processes 3 [0153] and 5 [0454], where FIG. 01 links into the self-iterating cycle of FIGS. 02-05. Finally, FIG. 05 completes the self-iterating cycle of FIGS. 02-05 through processes 6-9 [0581, 0582, 0590, 0568/0595], in which exosomes [0554, 0569] deliver their SIF-construct complexes [0566, 0567] to target cells [0510], where they can then replicate [0568] before being delivered [0346, 0366/0367, 0369, 0582] to other cells [0510] as the cycle (FIGS. 02-05) starts again. In this way, DNA, RNA, and protein transfer to an entire cell culture, tissue, organ, organ system, or even organism may take place for experimental, research, therapeutic, or diagnostic purposes.

What is claimed is:

1. A method for generating a Self-Iterating Factor (SIF) which functions as an intra-luminal vesicle translocation enabler within a viral vector complex involving a genetic construct of exosomal shuttle DNA, the method comprising:
a. generating a Vaccinia viral construct containing genetic DNA loci that encode an intra-luminal vesicle translocation factor known as a SIF at one locus and allow said SIF to bind to another locus;
b. transducing a cell with a virally packaged SIF construct for cytoplasmic unloading and cytoplasmic transcriptional activation;
c. entering the genetic construct into cytoplasma of the cell;
d. translocating the SIF-construct complex into an intra-luminal vesicle (ILV) of a multi-vesicular body (MVB) with the SIF;
e. containing the SIF-construct complex within the ILV until it is released into the extracellular space as an exosome containing exosomal shuttle DNA (esDNA); and
f. fusing the exosome with a target cell; and releasing the SIF-construct complex into the cytoplasm to transfect the target cell.

2. The method of claim 1 additionally comprising the step of starting again at step 1a and proceeding through step 1e.

3. The method of claim 1, including a genetic construct with one or more origins of replication derived from a same species as the target cell.

4. The method of claim 3, wherein the self-iterating factor is introduced to an epithelial layer of a tissue and a self-iterative process of esDNA transfer results in a genetic construct permeating the basal lamina as well as one or more of the surrounding tissues, organs, and organ systems.

5. The method of claim 4 wherein the genetic construct resides and replicates in the basal lamina, causing epithelial cell sheets to contain the genetic construct of exosomal shuttle DNA.

6. The method of claim 5, in which the genetic construct is used to treat a disease of epithelial tissue via gene therapy.

7. The method of claim 6 wherein the disease comprises cystic fibrosis with regard to epithelial lung tissue.

8. The method of claim 7, in which the self-iterating vector reaches the bloodstream, from which it seeks out and heals or destroys cancer cells by means of a targeted ligand which causes the vector to only bind to cancerous cells.

9. The method of claim 1, wherein the SIF is derived from one or more of: aspartic protease cathespin D, protein phosphatase 2A, protein kinase C-2, calpain, neutral sphingomyelinase, hsc70, Alix, TfR, Hrs, P1KFYVE, Annexin-11, Tsg101, LBPA, lectin, Pme117, and lactadherin acting as candidate factors involved in ILV translocation.

10. The method of claim 1, wherein the SIF-construct complex facilitates the deletion of one strand of the DNA in a region where the remaining strand is complementary to a known housekeeping esRNA, such that the SIF-construct complex hitchhikes a ride to another cell as a DNA-RNA hybrid.

11. The method of claim 5 in which one or both of the self-iterating vector, and the SIF-construct complex involving a genetic construct of exosomal shuttle DNA, is used to spread a genetic construct throughout essentially the entirety of a multi-cellular organism, such that one or more of the following is altered: the organism's genome, transcriptome, and proteome.

12. The method of claim 12 wherein the spread of a genetic construct throughout essentially the entirety of a multi-cellular organism facilitates one or more of experimental, research, therapeutic, and diagnostics.
